(19) United States
(12) Patent Application Publication
(10) Pub. No.: US 2007/0027434 A1
(43) Pub. Date: Feb. 1, 2007

(54) Adhesive face plate for an ostomy appliance, an ostomy appliance and a method for cleaning a face plate

(76) Inventors: Ole Pedersen, Strand (DK); Jan Wohlgemuth, Horsholm (DK); Michael O. Karlsson, Helsingor (DK)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/558,960

(22) PCT Filed: Jun. 1, 2004
(86) PCT No.: PCT/DK04/00378

§ 371 Date: Apr. 4, 2006

(30) Foreign Application Priority Data
Jun. 2, 2003 (DK) PA 2003 00815
Dec. 22, 2003 (DK) PA 2003 01912

(51) Int. Cl. A61F 5/44

(52) U.S. Cl. 604/333; 604/338

(57) ABSTRACT

An ostomy appliance comprising an adhesive face plate comprising an adhesive layer 2, a release sheet 3 and a carrier sheet 4 attached to a coupling ring 6 by means of a plastic film strip 9 forming a filter pouch together with a film strip 12 having a flatus gas venting aperture 18 covered by a filter 17, the filter pouch 9, 12 communicating through an aperture 20 in the film 9 with the interior of an ostomy bag 6b attached to the face plate by means of a coupling ring 6a engaging the coupling ring 6, an intermediate film 21 subdividing the filter pouch 9, 12 into two compartments 22 and 23 in flow communication through an aperture 24 in the film 21, the filter 17 being located in the compartment 23 such flatus gas flows from the interior of the bag 6b through aperture 20 into compartment 22, through aperture 24 into compartment 23, through filter 17 and out through aperture 18.

The present invention relates to an adhesive face plate for securing an ostomy bag to the skin of an ostomy patient, said faceplate comprising a flexible plate with a bodyside or proximal surface and a distal surface and comprising a layer of a skin friendly adhesive material and a stoma receiving aperture for receiving a stoma of said patient, generally annular face plate coupling means having an inner and an outer periphery and adapted for fixedly or releasably engaging corresponding bag coupling means of said ostomy bag, said face plate coupling means being secured to said flexible plate with the plane of said face plate coupling means generally parallel to the plane of said flexible plate and generally concentric with said aperture for receiving said stoma, and a filter for sing flatus gas.

As the de-odorising filter is relatively expensive, it is desirable to re-use the filter in connection with several consecutive disposable ostomy bags by attaching the filter to the face plate instead of to the ostomy bag such that the filter is not disposed of together with the full ostomy bag.

U.S. Pat. No. 4,232,672 discloses a face plate of the type in reference where a face plate coupling means in the form of a ring is provided with a lateral projection comprising a filter chamber containing filters communicating with two flatus gas passages extending through the coupling ring to the interior periphery thereof. Manually operable valve means allow flatus gas to flow through said passages and through the filters. This is a relatively complicated and expensive structure requiring manual action to vent the flatus gas. The dimension of the coupling ring perpendicular to the plane thereof is also relatively large so as to accommodate the venting mechanism, and thus the face plate is relatively bulky, which is undesirable.

U.S. Pat. No. 4,826,495 also discloses a face plate of the type in reference having a relatively thick retainer plate having an annular projection containing an annular duct communicating with the interior of an ostomy bag attached to the coupling ring through flatus gas inlet apertures in said annular projection. The annular duct communicates with a filter arranged in a recess provided in the body of the retainer plate. This is also a relatively expensive and complicated structure that also is relatively bulky.

It is a main object of the invention to provide an adhesive face plate with an integrated flatus gas de-odorising filter that is relatively simple to manufacture, is relatively inexpensive and has a maximum dimension perpendicular to the plane of the coupling ring that is relatively small such that the entire face plate is not unduly bulky (low profile).

According to the invention, this object is obtained by providing one or more flatus gas passages extending between said face plate coupling means and said flexible plate from said inner periphery to said outer periphery and through said filter.

Hereby, the coupling means is not affected, and the flatus gas passages may be provided without requiring expensive and bulky interior passageways in the coupling means.

In the currently preferred embodiment of the invention, the face plate coupling means and the bag coupling means are mutually matching coupling rings adapted for releasably engaging one another.

Alternatively, the face plate coupling means and the bag coupling means are mutually matching surfaces adapted for being releasably or fixedly adhered to one another by means of an adhesive or fixedly adhered to one another by means a heat seal seam.

According to the invention, the face plate may further comprise a compartment having an exterior portion located outside said outer periphery and an interior portion located between said coupling means and said flexible plate, a flatus gas outlet being provided in a wall of said exterior portion, said compartment forming part of said flatus gas passage, said filter being located within said compartment such that said flatus gas outlet is obstructed by said filter.

Hereby, a flatus gas filter housing is provided without requiring bulky and expensive modifications of the coupling ring.

In the currently preferred embodiment of a face plate according to the invention said exterior portion of said compartment is defined by a first film strip of plastic material, preferably adapted to define a pouch.

Hereby, a particularly inexpensive, low profile and easily manufactured filter housing having small space requirements is provided.

In another aspect, the present invention relates to an adhesive face plate for securing an ostomy bag to the skin of an ostomy patient, said faceplate comprising a flexible plate with a bodyside or proximal surface and a distal surface and comprising a layer of a skin friendly adhesive material and a stoma receiving aperture for receiving a stoma of said patient, generally annular face plate coupling means having an inner and an outer periphery and adapted for fixedly or releasably engaging corresponding bag coupling means of said ostomy bag, said face plate coupling means being secured to said flexible plate with the plane of said face plate coupling means generally parallel to the plane of said flexible plate and generally concentric with said aperture for receiving said stoma, a filter for de-odorising flatus gas, a flatus gas venting compartment in the form of a pouch or bag made of a first film of plastic material and extending outward beyond said outer periphery, said first film being provided with a flatus gas outlet aperture, and a flatus gas passage extending from said inner periphery into the interior of said venting pouch and through both said filter and said flatus gas outlet aperture, said filter being arranged such that substantially all said flatus gas vented through said flatus gas aperture flows through said filter.

Hereby, a simple, low profile and inexpensive face plate with integrated filter is provided.

The flatus gas passage may extend between said coupling means and said flexible plate or it may extend at least partly through a conduit extending through a portion of said coupling means from a conduit inlet to a conduit outlet.

In the latter case one edge of said pouch may by attached to a body side or proximal surface of said coupling means facing said flexible plate and the opposed edge of said pouch may be attached to a distal surface of said coupling means facing away from said flexible plate, said conduit outlet being located between said proximal and distal surfaces such that flatus gas exiting from said conduit enters said pouch.

In a yet further aspect, the present invention relates to an ostomy appliance for receiving human stomal discharge comprising in combination an adhesive face plate according to the invention and an ostomy bag for receiving human stomal discharge releasably or fixedly attached to said face plate.

In the currently preferred embodiment of the invention obstructing means for hindering or preventing stomal fluids and liquids entering said flatus gas passage is provided adjacent the inner periphery of the face plate coupling means and preferably the obstructing means comprises an elongate body of a gas permeable, preferably hydrophobic material such as open cell foam or non-woven material, said body having an arcuate, preferably closed loop and preferably annular shape and being located between said face plate coupling means and said stoma receiving aperture.

Preferably, the elongate body is attached to the ostomy bag and/or to the bag coupling means such that removal of the bag entails removal of the elongate body such that cleaning of the face plate when replacing the ostomy bag is reduced or completely eliminated.

In a yet further aspect, the invention relates to a method of cleaning an adhesive face plate for securing an ostomy bag having an opening for receiving stomal discharge to the skin of an ostomy patient, the faceplate comprising a flexible plate with a bodyside or proximal surface and a distal surface and comprising a layer of a skin friendly adhesive material and a stoma receiving aperture for receiving a stoma of said patient, and generally annular face plate coupling means having an inner periphery generally concentric with and spaced from the edge of said aperture for receiving said stoma, a generally annular face plate distal surface being defined between said inner periphery and said edge, said face plate coupling means being adapted for releasably engaging corresponding bag coupling means of said ostomy bag such that said bag opening is in register with said face plate aperture, the method comprising the steps of providing a separate closed loop, preferably annular elongate body having dimensions allowing said body to be located within said inner periphery and abutting said annular face plate surface, attaching said body to said ostomy bag, attaching said ostomy bag to said face plate by engaging said face plate coupling means with said bag coupling means such that said body is located within said inner periphery and abutting said annular face plate surface, and removing said ostomy bag together with said body by disengaging said face plate coupling from said bag coupling means.

In a final aspect, the invention relates to an ostomy appliance for receiving human stomal discharge from an ostomy patient and comprising in combination an adhesive face plate with a stoma receiving aperture for receiving a stoma of said patient and an ostomy bag with an opening for receiving said stomal discharge for receiving said stomal discharge releasably attached to said face plate, the face plate comprising a flexible plate having a bodyside or proximal surface and a distal surface, a layer of a skin friendly adhesive material, and generally annular face plate coupling means having an inner periphery generally concentric with and spaced from the edge of said aperture for receiving said stoma, a generally annular face plate distal surface being defined between said inner periphery and said edge, said face plate coupling means being adapted for releasably engaging corresponding bag coupling means of said ostomy bag such that said bag opening is in register with said face plate aperture, the ostomy appliance further comprising a separate closed loop, preferably annular elongate body attached to said ostomy bag and located within said inner periphery and abutting said annular face plate surface when said face plate coupling means are engaged with said bag coupling means.

In the following, the invention will be explained more in detail with reference to various embodiments thereof shown, solely by way of example, in the accompanying drawings, where

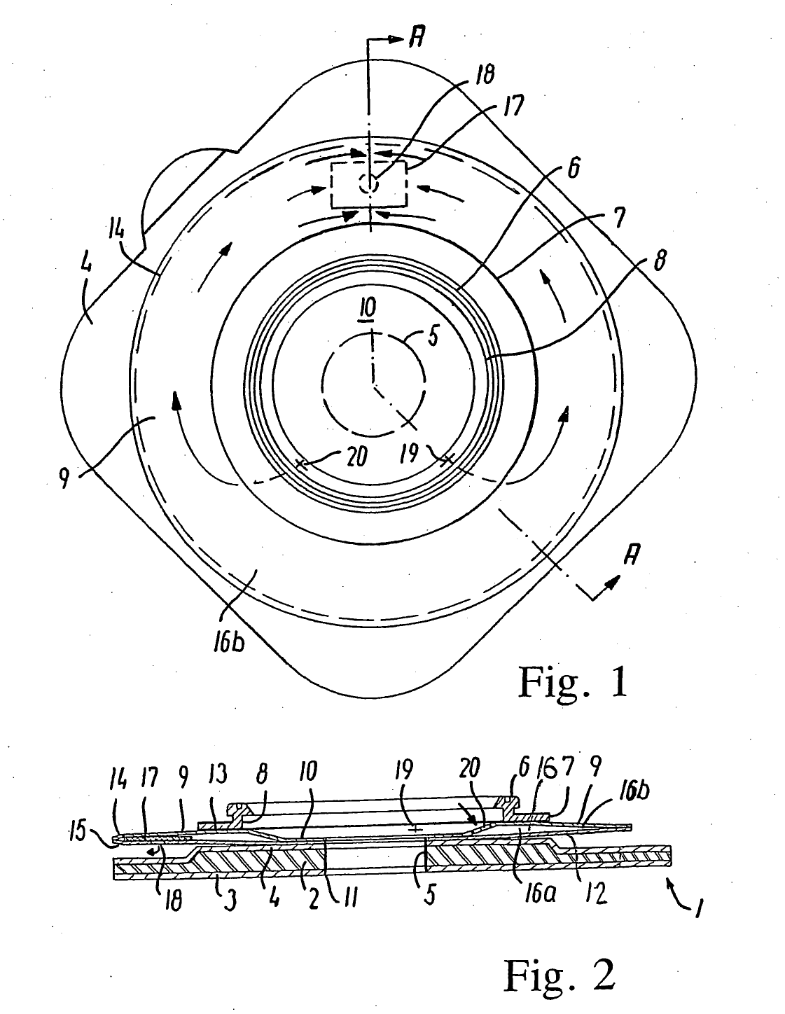

FIG. 1 is a diagrammatic top plan view of a first embodiment of a face plate according to the invention,

FIG. 2 is a cross section of the embodiment of FIG. 1 taken along line A-A,

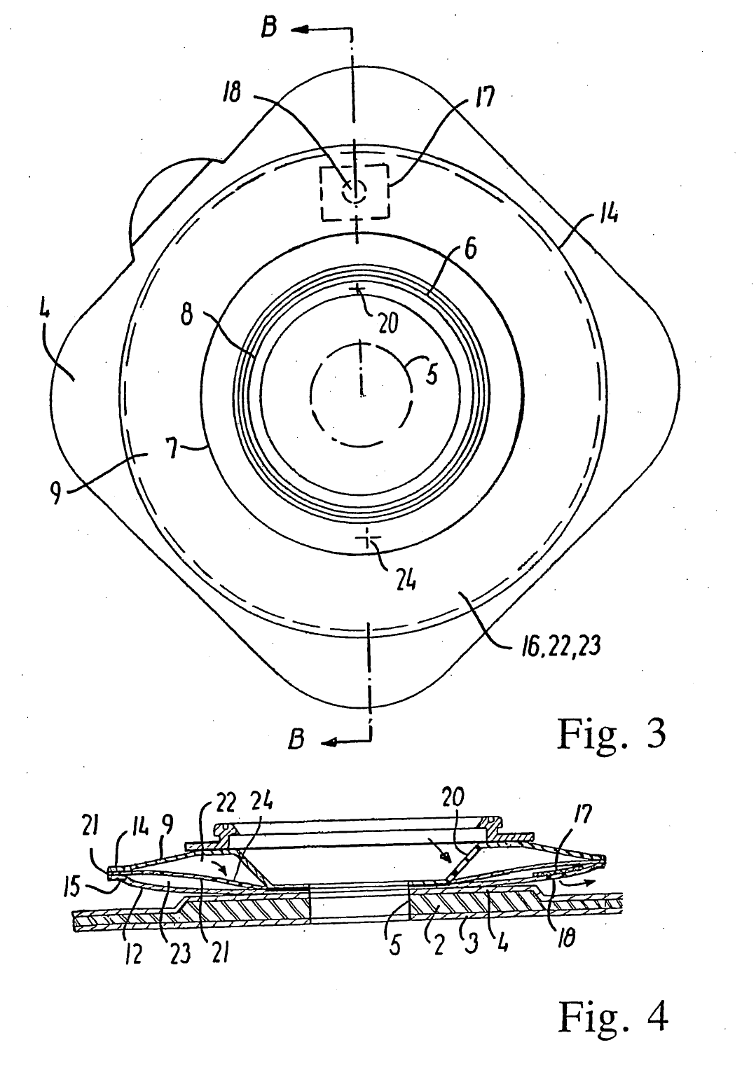

FIG. 3 is a diagrammatic top plan view of a second embodiment of a face plate according to the invention,

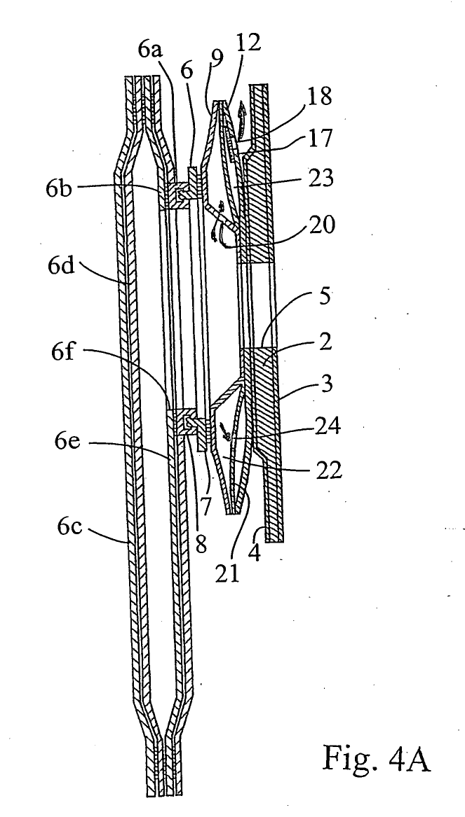

FIG. 4 is a cross section of the embodiment of FIG. 2 taken along line B-B,

FIG. 4A is a cross sectional view corresponding to FIG. 4 of the face plate of FIGS. 3-4 releasably connected to an ostomy bag for collecting human discharge,

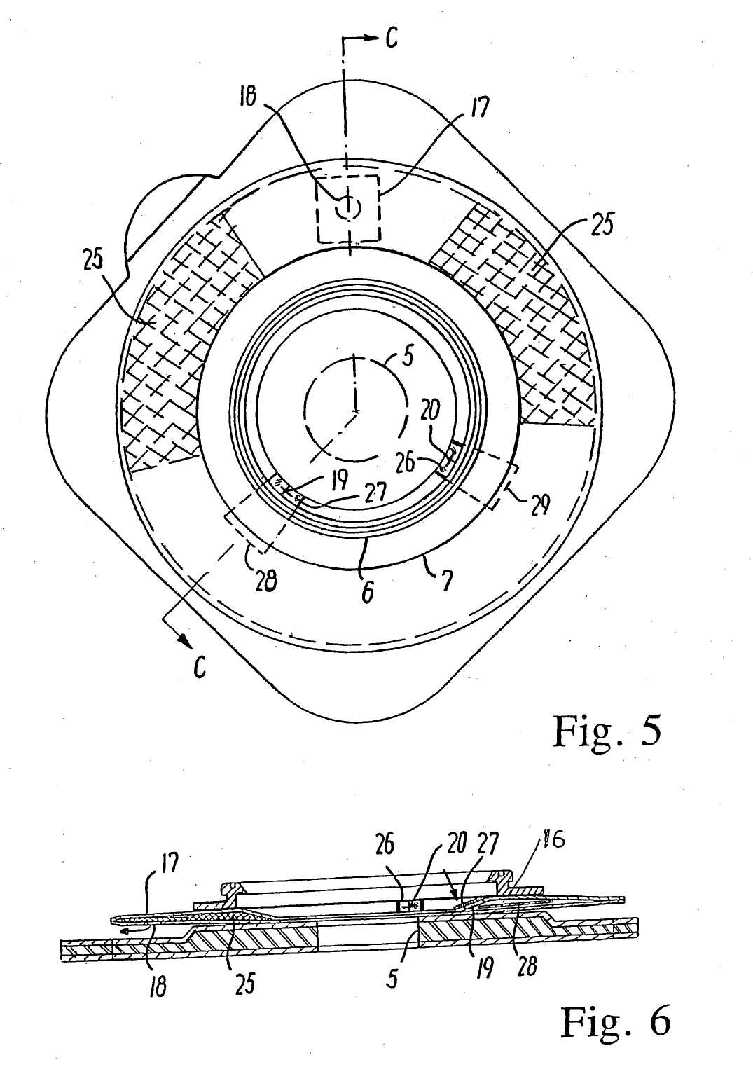

FIG. 5 is a diagrammatic top plan view of a third embodiment of a face plate according to the invention,

FIG. 6 is a cross section of the embodiment of FIG. 5 taken along line C-C,

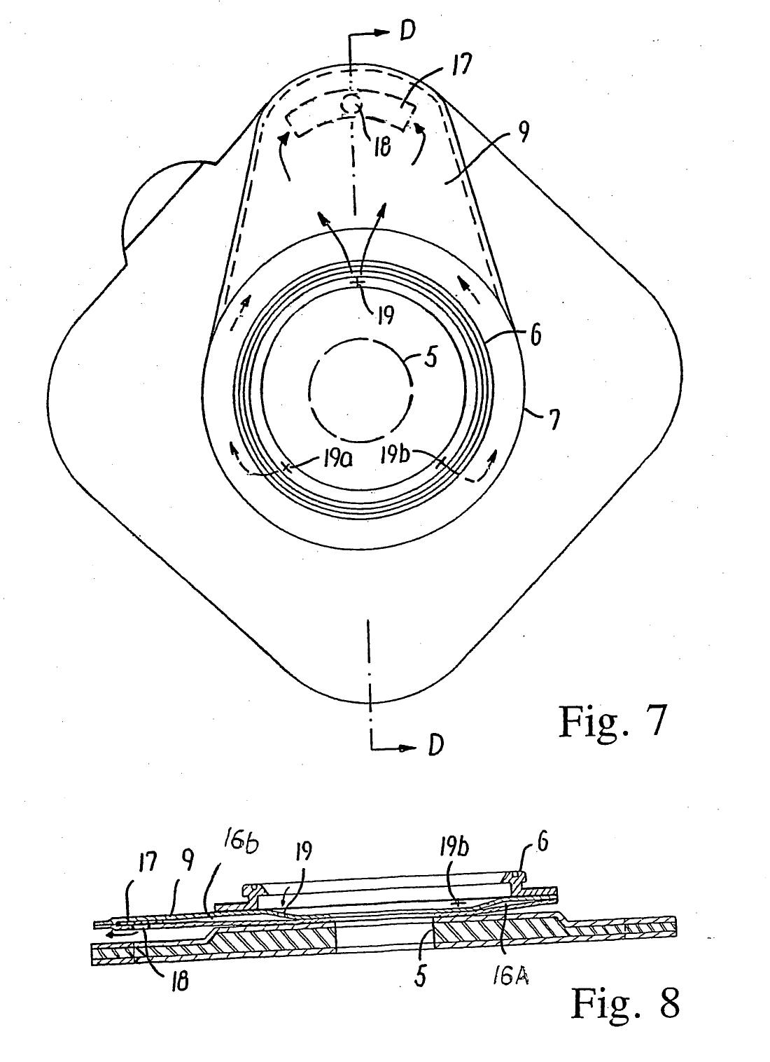

FIG. 7 is a diagrammatic top plan view of a fourth embodiment of a face plate according to the invention,

FIG. 8 is a cross section of the embodiment of FIG. 7 taken along line D-D,

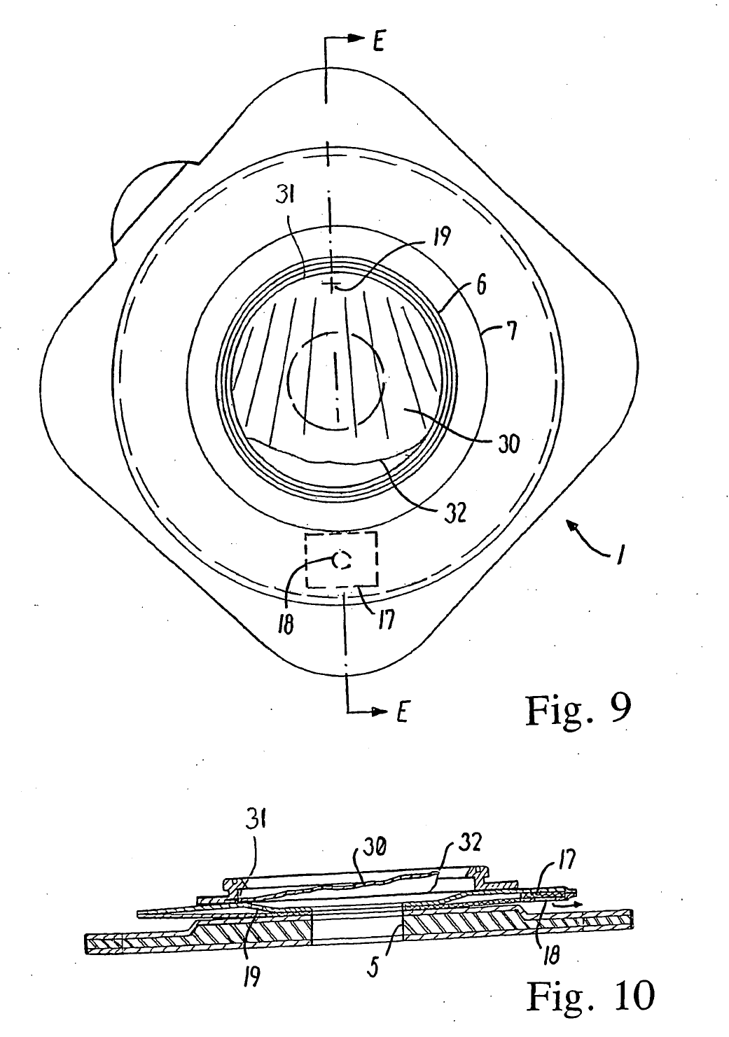

FIG. 9 is a diagrammatic top plan view of a fifth embodiment of a face plate according to the invention,

FIG. 10 is a cross section of the embodiment of FIG. 9 taken along line E-E,

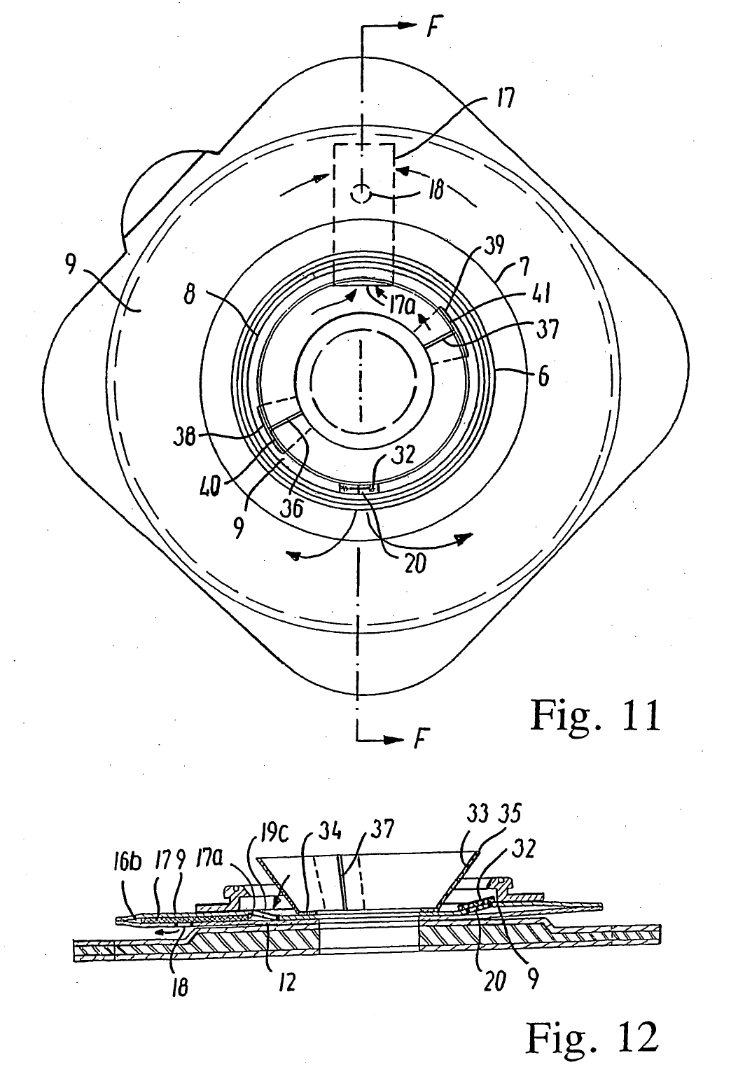

FIG. 11 is a diagrammatic top plan view of a sixth embodiment of a face plate according to the invention,

FIG. 12 is a cross section of the embodiment of FIG. 11 taken along line F-F,

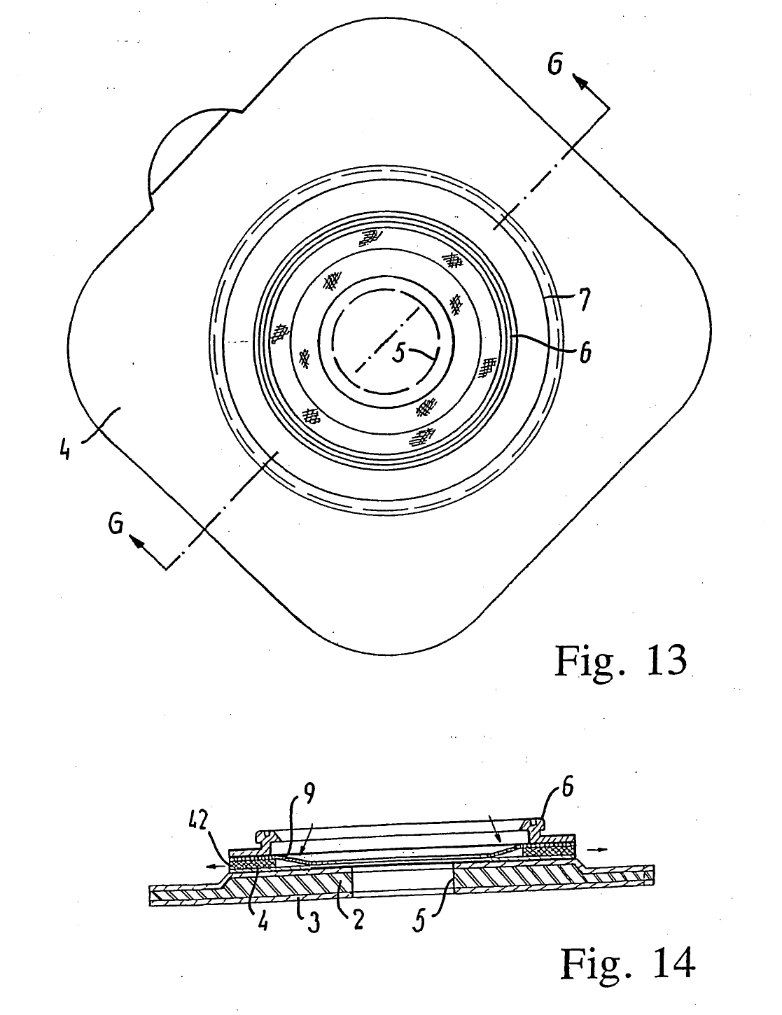

FIG. 13 is a diagrammatic top plan view of a seventh embodiment of a face plate according to the invention,

FIG. 14 is a cross section of the embodiment of FIG. 13 taken along line G-G,

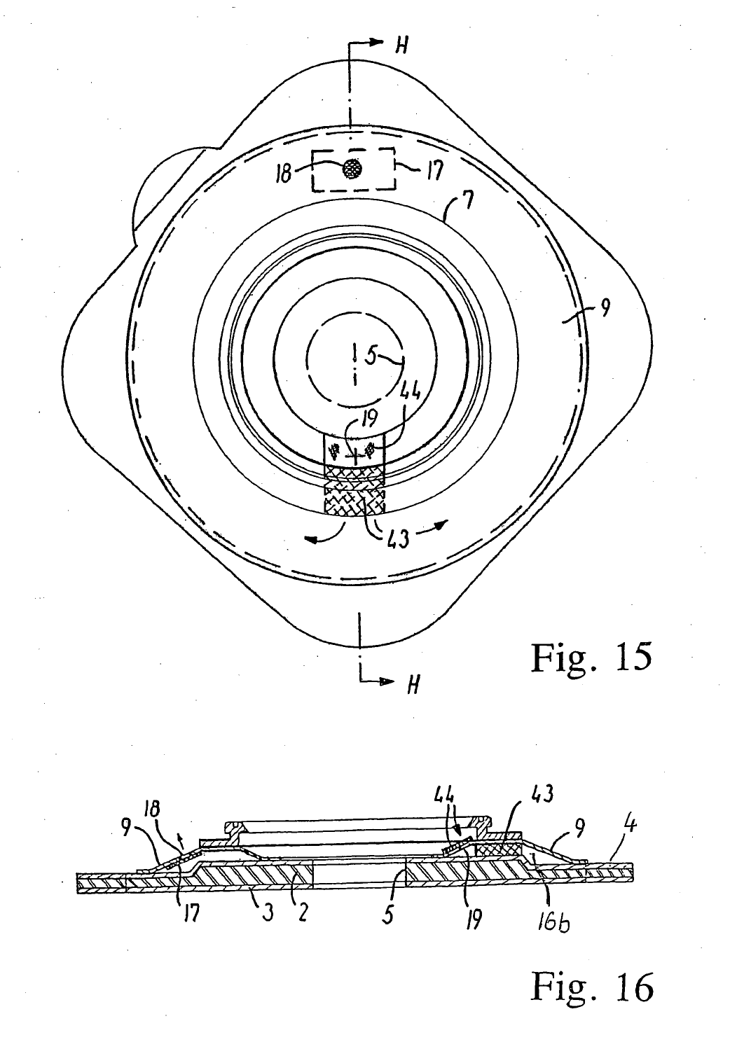

FIG. 15 is a diagrammatic top plan view of an eighth embodiment of a face plate according to the invention,

FIG. 16 is a cross section of the embodiment of FIG. 15 taken along line H-H,

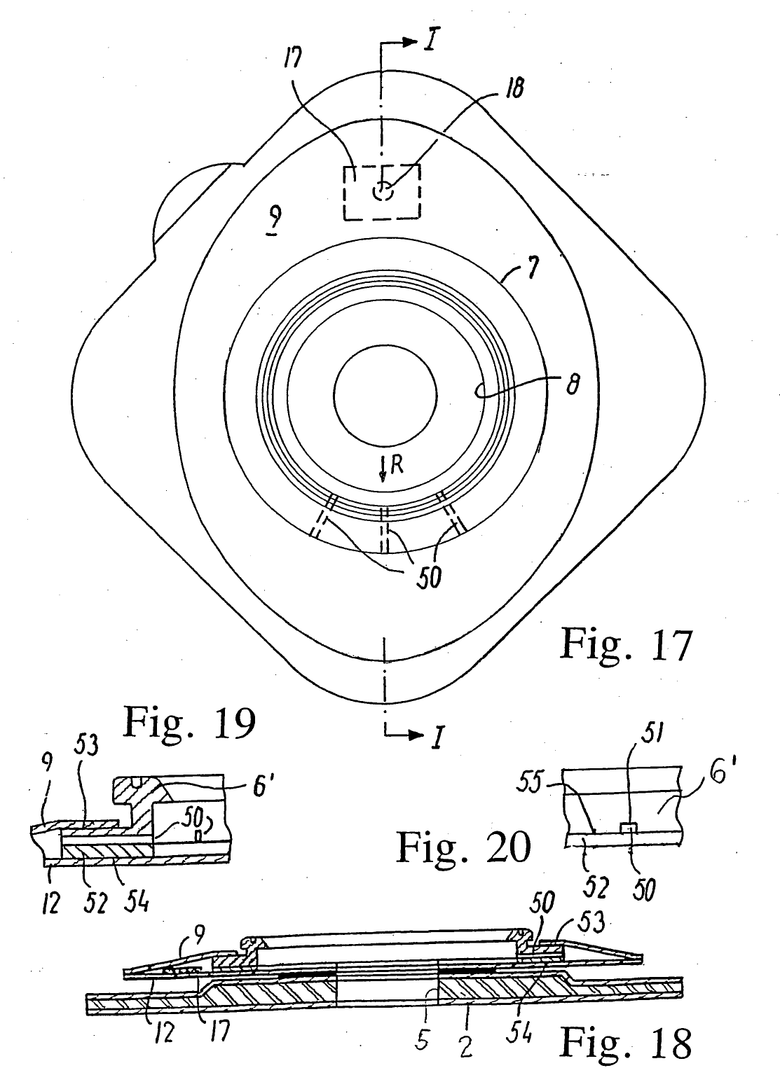

FIG. 17 is a diagrammatic top plan view of a ninth embodiment of a face plate according to the invention,

FIG. 18 is a cross section of the embodiment of FIG. 17 taken along line I-I,

FIG. 19 is an enlarged scale view of the cross section in FIG. 18 through the coupling ring,

FIG. 20 is an enlarged scale broken away elevational view seen in the direction of the arrow R in FIG. 17,

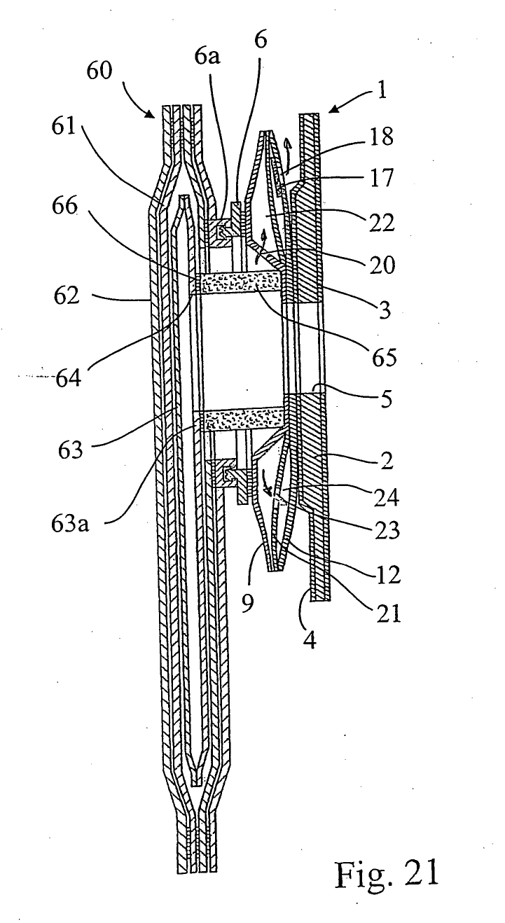

FIG. 21 is a cross sectional view corresponding to FIG. 4A of a further embodiment of an ostomy device according to the invention with an inner disposable pouch within an outer pouch,

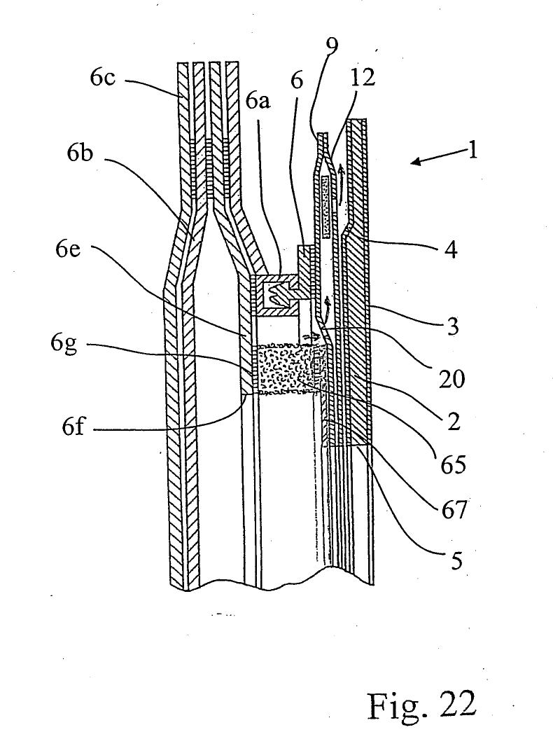

FIG. 22 is a cross sectional, broken-away view of the top portion of a further embodiment of an ostomy device according to the invention,

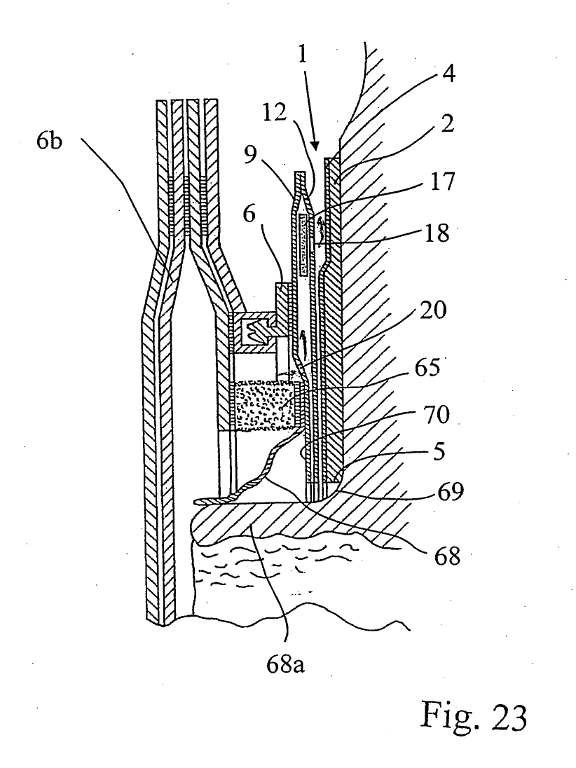

FIG. 23 is a cross sectional broken away view of the top portion of a further embodiment of an ostomy device according to the invention,

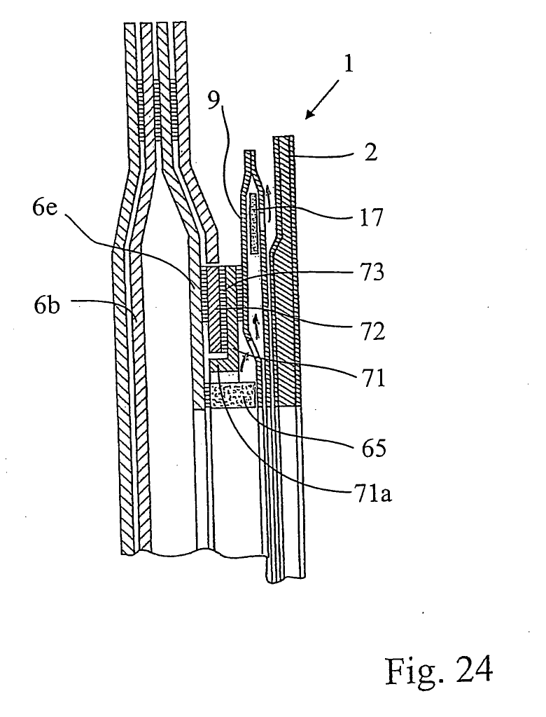

FIG. 24 is a cross sectional broken away view of the top portion of a further embodiment of an ostomy device according to the invention with an adhesive releasable attachment of the ostomy pouch to the face plate,

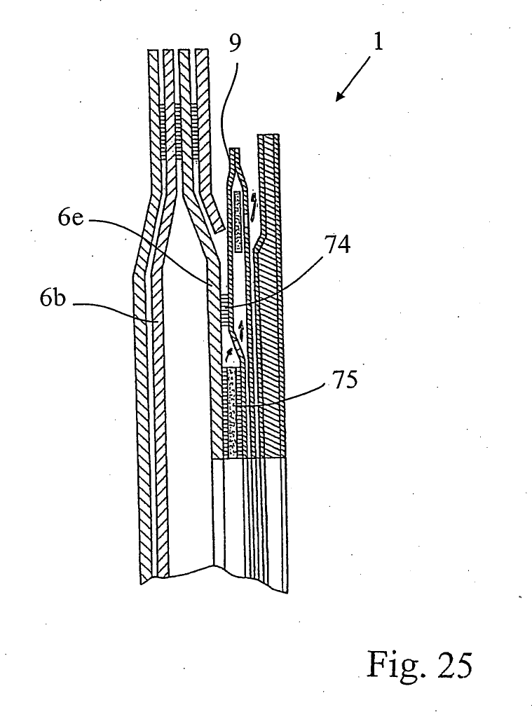

FIG. 25 is a cross sectional broken away view of the top portion of a further embodiment of an ostomy device according to the invention where the ostomy pouch is permanently attached to the face plate, a so-called one-piece ostomy appliance,

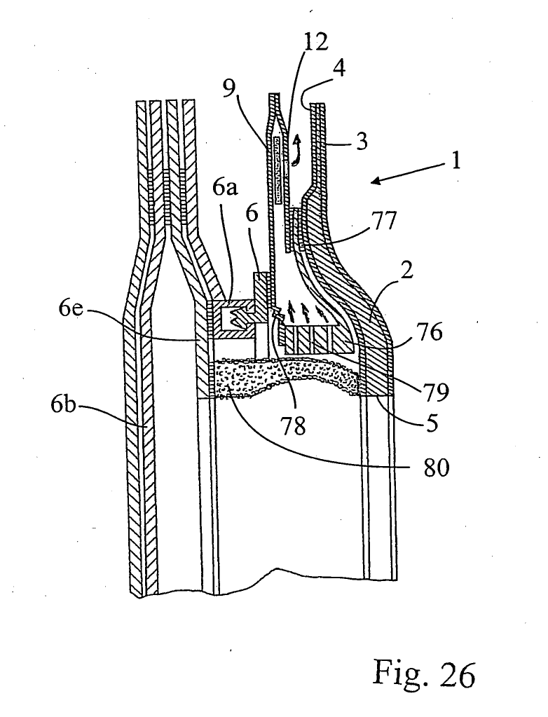

FIG. 26 is a cross sectional broken away view of the top portion of a further embodiment of an ostomy device according to the invention where the face plate is convex for being adapted to a depression in the peristomal skin surface of a user of the ostomy device,

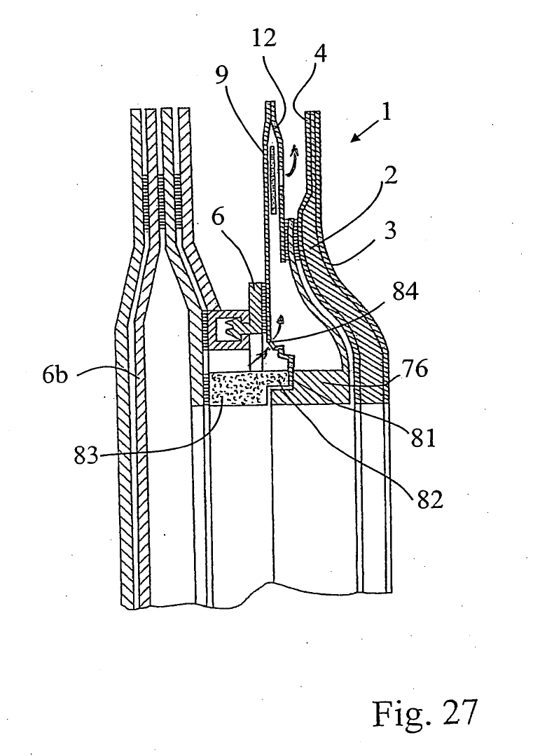

FIG. 27 is a cross sectional broken away view of the top portion of a further embodiment of an ostomy device according to the invention with a convex face plate,

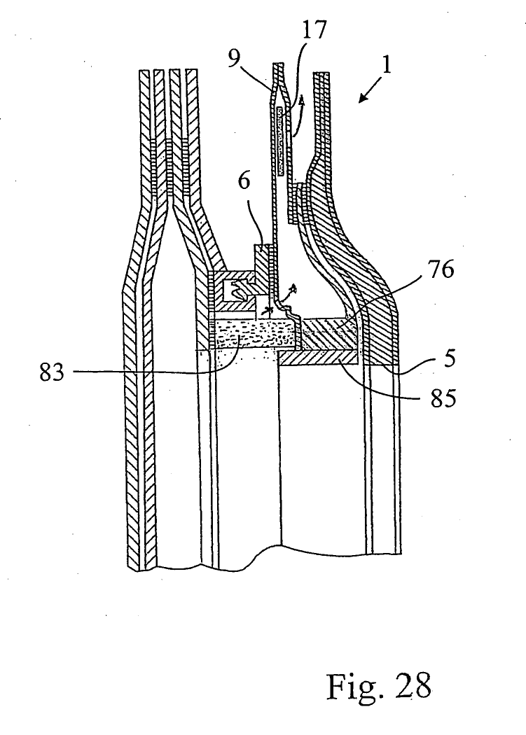

FIG. 28 is a cross sectional broken away view of the top portion of a further embodiment of an ostomy device according to the invention with a convex face plate, and

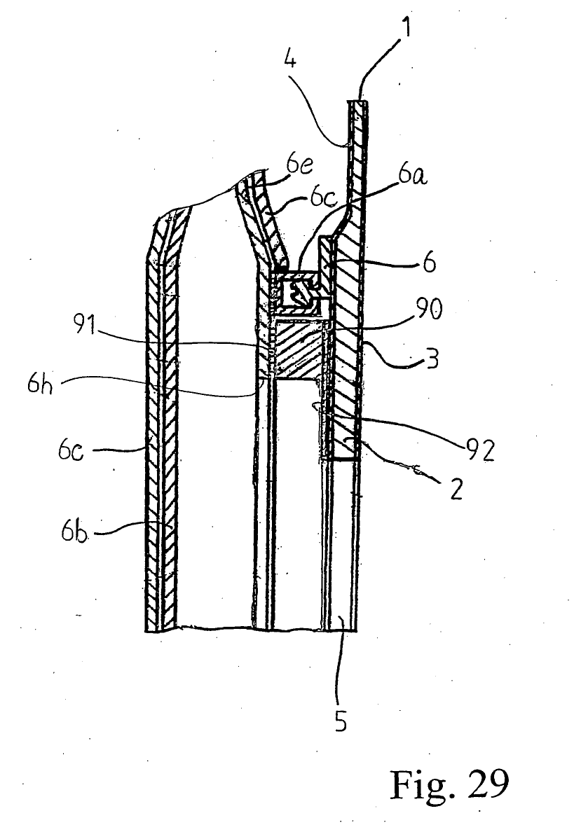

FIGS. 29-31 are cross-sectional broken away views of three embodiments of a two-piece ostomy appliance according to the invention with means to reduce the cleaning of the face plate when replacing the ostomy bag of the appliance.

In the drawings, similar elements of the different embodiments are referred to by the same reference numerals.

All the embodiments of a faceplate according to the invention are referred to generally by the numeral 1 and comprise a layer 2 of a pressure-sensitive skin friendly adhesive.

The adhesive layer 2 may be formed of any suitable pressure-sensitive adhesive commonly used for securing the faceplates of ostomy appliances to the peristomal skin surfaces of a wearer. For example, a hypoallergenic medical-grade acrylic adhesive may be used. However, it is preferable that the adhesive layer be formed of a soft, skin friendly hydrocolloid-containing adhesive material that is capable of absorbing moisture and has both wet and dry tack. Such a material is commonly referred to as a skin barrier composition and typically comprises a continuous elastomeric adhesive phase having hydrocolloid particles dispersed throughout the continuous phase. Initial tack, usually referred to as “dry tack,” is provided by the continuous phase but, because such a composition is occlusive or non-breathable, adherence to the skin would be disrupted by perspiration and by liquid stomal discharge if it were not for the dispersed hydrocolloids which absorb fluids and thereby maintain and possibly enhance adhesive attachment to the skin. U.S. Pat. No. 4,551,490 and other references disclose that suitable water-absorbing and swellable hydrocolloid gums may include sodium carboxymethylcellulose, pectin, gelatin, guar gum, locust bean gum, and the like. The elastomers used in the continuous phase may be polyisobutylene, natural rubber, silicone rubber, acrylonitrile rubber and other elastomers known in the art to have similar properties.

A silicone treated release sheet 3 of plastic material is applied to the body side or proximal surface of the adhesive layer 2 for protecting the adhesive layer 2 until the faceplate is to be applied to the skin of a wearer thereof.

A carrier sheet 4 of heat sealable plastic material is attached to the distal surface of the adhesive layer 2. An aperture 5 for receiving a stoma of a wearer of the face plate is provided in the layer 2 and in the sheets 3 and 4.

A coupling ring 6 of a flexible plastic material and having an outer periphery 7 and an inner periphery 8 is adapted for engaging a corresponding coupling ring 6a heat sealed on an ostomy bag 6b covered by a comfort layer 6c of non-woven material (FIG. 4A) for securing the bag to the wearer such that stomal material may be collected in the bag when the faceplate 1 with the bag secured to the faceplate is adhered to the peristomal skin of the wearer.

Referring now to FIGS. 1-2, the coupling ring 6 is secured to the adhesive layer 2 by means of a first annular strip 9 of film of a heat sealable plastic material having one edge 10 attached by heat sealing to one edge 11 of a second annular strip 12 of film of a heat sealable plastic material which edge 11 in turn is attached to the carrier sheet 4 by heat sealing. The coupling ring 6 is attached to the film strip 9 at 13 by heat sealing whereby the coupling ring 6 is flexibly attached to the adhesive layer 2.

Edges 14 and 15 of the strips 9 and 12, respectively, spaced outwardly from the edges 10 and 11, respectively, are heat sealed to one another such that an annular compartment or chamber 16 is formed having an interior portion 16a located between the coupling ring 6 and the adhesive layer 2 and an exterior portion 16b in the form of a pouch located outside the exterior periphery 7 of the coupling ring 6. The film strips 9 and 12 may be integral such that the heat sealing of edges 14 and 15 may be avoided.

A flatus gas de-odorising filter 17 is arranged in the exterior portion 16b of the compartment 16 and is adhered or heat sealed to film strip 12 such that it covers a flatus gas outlet aperture 18 provided in film strip 12. Two (or more) spaced flatus gas inlet apertures 19 and 20 are provided in the portion of the film strip 9 located inside the inner periphery 8 of the coupling ring 6. The inlet apertures 19 and 20 are located remote from the outlet gas aperture 18. The inlet apertures 19 and 20 are made by cutting a cross-shaped incision in the film strip 9.

In use, the release sheet 3 is removed from the adhesive layer 2, and the proximal surface of the layer 2 is applied to the peristomal skin of a wearer such that the stoma is received in the aperture 5. A collecting ostomy bag is then attached to the faceplate 1 by engaging the coupling ring of the bag with the matching coupling ring 13.

Stomal material consisting of solid material, liquid and flatus gas is then discharged from the stoma into the collecting or ostomy bag. The solid material and liquid falls down into the bag while the flatus gas is led out of the bag through the flatus gas passages defined by the inlet apertures 19 and 20, the interior compartment portion 16a and the exterior compartment portion 16b, the filter 17 and the flatus gas outlet 18. The flow of the flatus gas is indicated by means of arrows.

The filter 17 is of the type disclosed in U.S. Pat. No. 6,506,184 hereby incorporated herein by reference. However, any other suitable type of de-odorising filter may be used.

In this embodiment and the embodiments shown in FIGS. 3-12 and FIGS. 17-20, the coupling ring 6 is flexibly attached to the flexible plate 2, 3, 4 with the advantages inherent in this design as explained and disclosed in co-owned U.S. Pat. Nos. 4,419,100 and 5,730,735 hereby incorporated herein by reference.

Referring now to FIGS. 3-4A, this embodiment is identical to the embodiment of FIGS. 1-2 except for the provision of an intermediate strip 21 of a film of plastic material attached by heat sealing or adhesion to the film strips 9 and 12 such that the compartment 16 is subdivided into two annular compartments 22 and 23, the filter 17 being located in compartment 23. An aperture 24 is provided in the film strip 21 at a location opposite the inlet aperture 20 such that the flow of flatus gas indicated by the arrow is forced to flow through the labyrinth defined by the compartment 22, the aperture 24 and the compartment 23 before reaching the filter 17 and exiting through the flatus gas outlet 18. If any stomal liquid or solid material enters the compartment 16 through the inlet aperture 20 the labyrinth created by interposing the film strip 21 will create a larger distance between the inlets 19 and 20 and the filter 17 such that the probability of any such liquid or solid material clogging the filter is reduced.

The ostomy bag 6b is a conventional bag comprising two film sheets 6d and 6e heat sealed to one another along the edges thereof to form a bag having an aperture 6f for receiving stomal discharge. The comfort layer 6c is heat sealed to the film walls 6d and 6e along the outer edge thereof.

The film 21 may be substituted by a layer of non-woven material or other material that is permeable to flatus gasses, but hinders flow of liquid through the layer. In such case it is not necessary to provide a flatus gas aperture 24.

Referring now to FIGS. 5-6, this embodiment is the same as the one in FIGS. 1-2 with the following exceptions:

open cell foam material 25 or other suitable porous material is interposed in the annular compartment 16 between the inlets 19 and 20 and the filter 17, the foam material preferably being hydrophobic.

strips 26 and 27 of a gas permeable, liquid impermeable material are adhered to the film strip 9 around the apertures 19 and 20, respectively, such that the strips 26 and 27 cover said apertures, and

strips 28 and 29 of non-woven material are arranged in the compartments 16 adjacent the inlet apertures 19 and 20, respectively, so as to function for ensuring that there is a space between the film strips 9 and 12 adjacent the inlet openings 19 and 20 even though the film strips 9 and 12 have a tendency to cling or adhere to one another because of moisture or other factors.

The open cell foam material 25 fills out a cross section of the chamber 16 and thus prevents any liquid or solid material entering the chamber 16 through the inlet openings from reaching the filter 17 while still allowing flatus gas to flow through the chamber 16 to the filter.

The strips 26 and 27 of breathable material perform the same function of allowing flatus gas to pass, but at the same time preventing liquid or solid material from entering the compartment 16.

The open cell foam material 25, the gas permeable, liquid impermeable film strips 26 and 27 and the spacing members 28 and 29 of non-woven material may be applied together or individually to all the other embodiments except the embodiment shown in FIGS. 13-14. The strips of non-woven material may extend along the entire circumference of the coupling ring.

Referring now to FIGS. 7-8, this embodiment is very similar to the embodiment in FIG. 1-2 except that the exterior portion or pouch 16b of the compartment does not extend around the entire outer periphery of the coupling ring 6, but only extends along a portion of said periphery. The inlet aperture 19 is shown close to the filter 17, but alternative apertures 19a and 19b could also be arranged opposite the filter relative to the coupling ring as in the FIGS. 1-2 embodiment. The flow of flatus gas in connection with the alternative apertures 19a and 19b is indicated with dotted line arrows.

The filter 17 is shown arcuate with the arcuate edges being impermeable to gas such that the flow of flatus gas into the filter takes place through the opposed ends of the filter. The filter 17 could also in this case be square or rectangular as in the FIGS. 1-2 embodiment.

Referring now to FIGS. 9-10, this embodiment is identical to the FIGS. 1-2 embodiment except for the addition of a curtain 30 made of a patch or strip of film of a plastic material having one edge 31 attached by heat sealing to the coupling ring 6 along part of the circumference hereof and the opposed edge 32 free.

The curtain 30 will tend to prevent or hinder stomal liquid or solids from entering the flatus gas inlet 19 when the face plate 1 with a pouch attached is applied to the patient such that the curtain 30 extends downwards when the patient is standing. The stomal material will fall into the pouch through the aperture defined by the free edge 32 and the flexible face plate 1.

This curtain may be applied to all the other embodiments shown in the drawings except the FIGS. 11-12 embodiment.

Referring now to FIGS. 11-12, in this embodiment, the filter 17 extends from the exterior compartment portion or pouch 16b between the coupling ring 6 and the flexible plate 2, 3, 4 beyond the inner periphery 8 of the coupling ring through an aperture 19c in the film strip 9 to an edge 17a located within said inner periphery.

A second gas inlet aperture 20 is located opposite the aperture 19c and is covered by a patch or strip of 32 of a gas permeable, liquid impermeable film material. The aperture 19c may also be covered by such a patch of gas permeable, liquid impermeable material.

A strip 33 of a film of plastic material has one edge 34 heat sealed to the film strip 9 while the opposed edge 35 is free such that an annular collar is formed within the inner periphery 8. The film strip 33 is provided with two transverse slits 36 and 37 and further strips 38 and 39, respectively, of a film of a plastic material are heat sealed at 40 and 41, respectively, to the strip 33 on one side of the slits 36 and 37, respectively.

Hereby, the collar 33 may be deformed outwards by pressure from stomal material so to cover the apertures 19c and 20 because of the deformability afforded by the slits 36 and 37 while the overlap of the strips 38 and 39 prevents or hinders that stomal material can be pressed through the slits 36 and 37, respectively.

This collar 33 may be applied to all the other embodiments shown in the drawings except the FIGS. 9-10 embodiment.

Referring now to FIGS. 13-14, in this embodiment an annular filter 42 is arranged between the coupling ring 6 and the carrier sheet 4 and is attached to the film strip 9 and the carrier sheet 4 either by heat sealing or by means of an adhesive. The annular film strip 9 is made of a heat sealable, gas permeable, liquid impermeable film. The construction of the filter 42 is such that it has sufficient shear strength to ensure that the coupling ring 6 is securely fastened to the carrier sheet 4 and thereby to the adhesive layer 2.

The flatus gas may flow out at all points along the coupling ring 6 whereby a particularly effective venting of the flatus gas is achieved. An annular filter pouch such as pouch 9, 12 in FIGS. 1-4A with a flatus escape aperture 18 may be arranged around annular filter 42 for preventing water from blocking filter 42 if the user of the ostomy device showers.

Referring now to FIGS. 15-16, in this embodiment the film strip 12 has been eliminated and the edge 14 of the film strip 9 is heat sealed to the flexible plate 2, 3, 4 such that the exterior portion 16b of the compartment is defined by the film strip 9 and the carrier sheet 4.

The flatus gas inlet aperture 19 is covered by a patch of gas permeable, liquid impermeable plastic film, and a spacer 43 in the form of a strip of open cell foam material is arranged adjacent the aperture 19.

The de-odorising filter 17 is adhered to the film strip 9 around a flatus gas outlet aperture 18 provided in the film strip 9.

Referring now to FIGS. 17-20, an alternative embodiment is shown, wherein the pouch is located in a different manner. The film strip 9 defining the pouch together with film strip 12 has one edge attached to the proximal surface 53 of the coupling ring 6′ while the film strip 12 is attached to the distal surface 54 of the coupling ring 6′ and to the carrier sheet 4. The flatus gas passage comprises one or more conduits 50 extending through the coupling ring from the interior periphery 8 to the outer periphery 7 such that the outlet of the or each conduit is located between the distal and proximal surfaces so that the gas passage extends between said edges of the film strip defining the pouch.

The conduit 50 may be provided without greatly increasing the dimension of the coupling ring perpendicular to the plane thereof. One way of providing said conduit 50 is illustrated in FIG. 20. A channel 51 is formed in the distal surface 55 of a coupling ring very similar to coupling ring 6 shown in FIGS. 1-16. A ring 52 is thereafter adhered to said distal surface 55 whereby the coupling ring 6′ provided with the conduit 50 is formed.

One advantage of this alternative embodiment is that the coupling ring 6′ with attached pouch 9, 12 may be manufactured separately and subsequently attached to the carrier sheet 4 by heat sealing the edge portion of film strip 12 to the carrier sheet.

The shape of the pouch 9, 12 is oval so as to save material for the film strip 9 and 12 and still have sufficient space for any spacing means adjacent the conduits 50 and for the filter 17.

It should be noted that alternatively the flatus gas passage may be provided in the adhesive layer 2 of the flexible plate 2, 3, 4. An elongate filter may also b_ embedded in the adhesive layer 2 extending from the stoma receiving aperture 5 to the outer periphery of the adhesive layer 2.

This gas passage in the adhesive layer may also be a groove provided in the surface of the adhesive layer 2 covered by the carrier sheet 3, the groove extending from the stoma receiving aperture 5 to an aperture in the carrier sheet 3, The de-odorising filter may be attached to the carrier sheet such that the flatus gas vented through the groove and the aperture is constrained to flow through the filter.

Referring now to FIG. 21, a face plate 1 identical to the one shown in FIGS. 3-4A, except that there are a number of apertures 20 distributed around the upper half circle of the film 9, is attached to an ostomy bag referred to generally by the numeral 60 and comprising an outer bag 61 of a liquid and gas impermeable plastic sheet material covered by a comfort layer 62 of a non-woven material.

An inner bag 63 made of plastic sheet material that is biodegradable and/or soluble under certain circumstances is arranged inside the outer bag 61 and is provided with an aperture 64 for receiving stomal discharge. This inner bag 63 is intended for being separated from the outer bag and disposed of by being flushed down a toilet when full of stomal discharge.

So as to avoid that liquid and/or semi-solids from the stomal discharge from entering the filter pouch 9, 12 through the apertures 24, an obstruction means for solids and liquids is provided in the form of a closed loop configured structure. Preferably, as shown in FIG. 21, the obstruction means takes the form of a ring 65 of resilient open cell foam material, preferably a biodegradable material, but also usefully of any other resilient or also semi-rigid or rigid material allowing flatus gasses to pass therethrough, but hindering or preventing stomal effluent solids and liquids from passing therethrough.

The ring 65 operates as a pre-filter and is arranged between the aperture 5 of the face plate 1 and the coupling rings 6, 6a to allow flatus gasses to pass through the obstruction means into the chamber 22 and through the filter 17 and out of the aperture 18, but to hinder solids and liquids from passing into the chamber 22 so that the filter 17 is not clogged by said solids and liquids.

The ring 65 is attached to the inner bag wall 63a around the aperture 64 by means of an annular seam 66 of adhesive or weld such that the ring 65 is removed together with the inner bag 63 and flushed down a toilet.

Ideally, the material of the ring 65 is compressible and the axial extent of the ring 65 is slightly larger than the distance between the inner bag wall 63a and the carrier sheet 4 when the coupling rings 6 and 6a are engaged such that in said engaged position of the coupling rings the ring 65 is slightly axially compressed such that a pressure is exerted by the ring 65 on the strip 9 and carrier sheet 4 to ensure that no solids and liquids may pass between the ring 65 and the face plate 1.

In use, the release sheet 3 is removed from the adhesive layer 2, and the proximal surface of the layer 2 is applied to the peristomal skin of a wearer such that the stoma is received in the aperture 5. The collecting ostomy bags 61 and 63 are then attached to the faceplate 1 by engaging the coupling ring 6a of the bags with the matching coupling ring 6.

Stomal material consisting of solid material, liquid and flatus gas is then discharged from the stoma into the inner bag 63. The solid material and liquid falls down into the inner bag 63 while the flatus gas is led out through the ring 65 and the flatus gas passages defined by the inlet apertures 24, the compartment portion 22, the aperture 24 and the compartment portion 23, the filter 17 and the flatus gas outlet 18. The flow of the flatus gas is indicated by means of arrows.

The filter 17 is of the type disclosed in U.S. Pat. No. 6,506,184 hereby incorporated herein by reference. However, any other suitable type of de-odorising filter may be used.

The coupling ring 6 is flexibly attached to the flexible plate 1 with the advantages inherent in this design as explained and disclosed in co-owned U.S. Pat. Nos. 4,419,100 and 5,730,735 hereby incorporated herein by reference.

When the inner bag 63 is full and is to be replaced by an empty bag, the coupling rings 6 and 6a are disengaged and a new set of inner and outer bags 61 and 63 with a new, clean pre-filter ring 65 is attached to the face plate 1. In this operation the ring 65 performs the further function of guiding the coupling ring 6a into engagement with the coupling ring 6, as the ring. 65 protrudes proximally beyond the coupling ring 6a.

Hereby a new pre-filter ring 65, unsoiled by stomal solids and liquids is provided such that the flatus gas again can flow unhindered by solids and liquids adhered to the inner surface of the ring 65 to the filter 17.

Furthermore, the ring 65 prevents stomal solids and liquids from soiling the coupling ring 6a allowing the outer bag 63 to be folded together and disposed of in a pocket or bag without emitting any odours.

The ring 65 also prevents stomal solids and liquid from contacting the coupling ring 6 of the face plate 1 as well as a major region of the distal surface of the face plate 1 located between the coupling ring 6 and the stoma receiving aperture 5. This renders the cleaning of the face plate when replacing the pouches 61 and 63 much easier and more effective as only the region between the inner surface of the ring 55 and the stoma received in the aperture 5 is to be cleaned. Reference is made to FIGS. 29-31 discussed below.

The material of the ring 65 may be any suitable porous, preferably at least slightly resilient, material such as open cell foam, non-woven material, textile, gauze and so on. The material is preferably hydrophobic and biodegradable or soluble under certain circumstances.

The currently preferred material for the ring 65 are fibres of 100% rayon coated with a paraffin wax (a paraffin emulsion Aurapel 374 supplied by the company Bayer Chemie). The ring has a thickness of approx. 9 mm and a weight of 35 oz/sqy and is supplied by the company High Tech Conversion.

The surface of the ring 65 facing the stoma receiving aperture 5 may be treated with a skin friendly product such as aloe vera or some other porous skin friendly cream such that any contact between the ring 65 and the stoma will bring the skin friendly material into contact with the stoma.

As shown most clearly in FIG. 22, the pre-filter ring 65 is attached to the proximal wall 6e of the bag 6b around the stoma receiving aperture 6f thereof by means of an adhesive seam or a heat seal 6g such that when replacing the bag with a new empty bag the ring 65 is also replaced as described above in connection with FIG. 21.

An annular plastic film 67 may be attached to the proximal surface of the ring 65 and be pressed against the face plate 1 by the ring 65. The film ring 67 prevents stomal material from soiling the region of the face plate 1 between the ring 65 and the stoma receiving aperture 5 such that when the pouch 6b is removed. the face plate 1 and coupling rings 6 and 6a are substantially clean. The film ring 67 may also be applied to the pre-filter 65 in FIG. 21.

Referring now to. FIG. 23, the embodiment shown therein is identical to the embodiment of FIG. 22 except that the annular plastic film in FIG. 23 lying flat against the carrier sheet 4 of the face plate 1, a sheath or cuff 68 of a plastic material is fastened to the pre-filter ring 65.

The sheath 68 abuts the circumferential surface of a stoma 68a received in the aperture 5 of the face plate 1 and thereby protects the stoma 68a and the peristomal skin surface 69 from contact with stomal material and also ensures that the distal surface region 70 of the face plate between the ring 65 and the stoma 69 is not soiled by stomal material such that when the pouch 6b with ring 65 and sheath 68 is removed for being replaced by a new pouch 6b with a new ring 65 with sheath 68, the necessity for cleaning the face plate is eliminated or dramatically reduced.

The material of the sheath may be any skin friendly material which preferably is plastically and/or elastically deformable.

Referring now to FIG. 24, in this embodiment the coupling means for releasably attaching the bag 6b to the face plate 1 comprises a flanged ring 71 of relatively rigid material and a planar ring 72 also of relatively rigid material releasably attached to one another by means of a layer of releasably adhesive material 73, the rings 71 and 72 being fixedly attached by heat sealing to the filter pouch film 9 and to the proximal bag wall 6e, respectively. The flange 71a of the ring 71 serves to guide the ring 72 when mounting a new bag 6b on the face plate 1.

Referring now to FIG. 25, a so-called one-piece ostomy appliance is shown where the bag 6b is fixedly attached to the face plate 1 by means of a heat seal or adhesive seam 74 between the filter pouch wall 9 and the proximal bag wall 6e.

A flat pre-filter ring 75 corresponding to ring 65 in FIGS. 21-25 is fixedly attached to the proximal bag wall 6e and to filter pouch wall 9 such that all flatus gas is forced to flow through ring 75 as indicated by the arrows.

Referring now to FIG. 26, a so-called convex face plate 1 for use by persons with depressed or recessed stomas is shown wherein the adhesive layer 2 is constrained to the proximally convex form shown by means of a relatively rigid convex ring 76 fixedly attached to the carrier sheet 3 by means of a heat seal seam 77.

The convex ring 76 is flexibly attached to the coupling ring 6 by means of the filter pouch film 9 that is provided with annular accordion-like folds 78 for allowing the coupling ring 6 to move relative to the convex ring 76 which facilitates coupling and uncoupling of the rings 6 and 6a for replacement of the bag 6b.

Flatus gas passages 79 are provided through the convex ring 76 for allowing flatus gas to pass into the filter pouch 9, 12 as indicated by the arrows. The passages 79 are provided evenly distributed along at least the upper half of the convex ring.

A pre-filter ring 80 corresponding to pre-filter ring 65 in FIGS. 21-23 is fixedly attached to the proximal pouch wall 6e and has a proximally tapering cross-section so as to reflect the reduced distance between the convex ring 76 and the stoma receiving aperture 5 such that a stoma received in the aperture does not come into too forceful contact with the ring 80.

Referring now to FIG. 27, another embodiment of a convex ostomy appliance is shown wherein the convex ring 76 is provided with an annular recess 81 for receiving an annular flange 82 of a pre-filter ring 83 corresponding to pre-filter ring 80 in FIG. 26.

In this embodiment, the flatus gas flows into the filter chamber 9, 12 through one or more apertures 84 in the film 9 in the region between the coupling ring 6 and the convex ring 76 as indicated by the arrows.

Referring now to FIG. 28, this embodiment is identical to FIG. 27 except that the pre-filter ring 83 does not have a flange and instead is kept in place on the convex ring 76 by means of an additional ring 85 fitting tightly inside the convex ring 76.

Referring now to FIG. 29, an ostomy appliance comprising a face plate 1 with an adhesive flexible plate 2, a release sheet 3, a carrier sheet 4 and a stoma receiving aperture 5, and an ostomy bag 6b attached to the face plate 1 by means of interlocking coupling rings 6 and 6a, is provided with means in the form of a separate, closed loop, preferably annular, axially elongated body for hindering or preventing contact between stomal discharge and the face plate distal surface and the coupling rings 6 and 6a.

In the FIG. 29 embodiment these means comprise an axially elongated body in the form of a ring 90 of a shape recoverable, resilient, compressible open cell or closed cell foam material or other material having similar properties fixedly attached to the ostomy bag proximal wall 6e around an opening 6h in said proximal wall 6e by means of an adhesive or heat sealing seam 91. The ring 90 is preferably hydrophobic.

An annular film 92 of plastic material is attached to the proximal surface of the ring 90 and extends to the edge of the aperture 5 such that the ring 90 and the film 92 protect substantially the entire area of the face plate between the coupling ring 6 and the aperture 5 from coming into contact with stomal discharge.

When the bag 6b is full of stomal discharge and is to be replaced, the coupling rings 6 and 6a are disengaged and the bag 6a with ring 90 and annular film 92 is removed. The necessity of cleaning the face plate 1 and coupling rings 6 and 6a after removal of the bag 6b and before attaching a new empty bag 6b is dramatically reduced or even totally eliminated.

The height of the ring 90 in the direction at right angles to the plane of the face plate 1, i.e. the axial dimension of the ring 90, should be slightly larger in an uncompressed state than the combined height of the coupling rings 6 and 6a so that the ring 90 is pressed against the face plate distal surface so that no stomal discharge can penetrate between the ring and the face plate.

The width of the ring parallel to the face plate plane may be so large that the ring itself covers the face plate surface between the coupling rings and the aperture 5. Even though the width of the ring parallel to the face plate plane is less than the distance between the coupling rings 6, 6a and the edge of the aperture 5 it is not necessary that the annular film 92 be provided, although in such case some cleaning of the face plate is normally necessary.

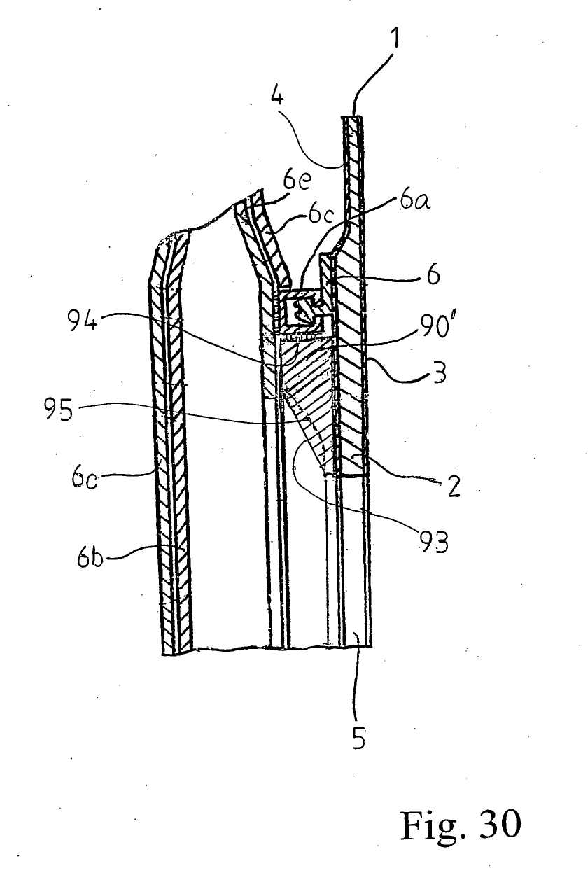

Referring now to FIG. 30, this embodiment is identical to the FIG. 29 embodiment except that the ring 90′ is so wide that it covers the entire area of the distal surface of the face plate between the coupling rings and the aperture 5, that the inwards facing surface 92 of the ring 90′ is oblique, and the ring 90′ is attached to the coupling ring 6a by means of an adhesive or heat seal seam 94.

The inwardly facing surface of the ring 90′ may be inwardly concave as indicated by the dotted line 95 such that the height or axial dimension of the ring 90′ in the region adjacent the aperture 5 is even smaller than in the embodiment with the oblique surface 93.

It is often that case that the individual ostomate adapts the contour of the face plate aperture 5 to the size and/or shape of his or her stoma by cutting the face plate with a scissors or a cutting tool. By reducing the height of the ring 90′ near the aperture 5 it is rendered easier for the individual ostomate to also adapt the inner edge of the ring 90′ correspondingly such that the ring 90′ does not press too much against the stoma when mounted on the face plate. In the FIG. 29 embodiment it is relatively easy to adapt the inner edge of the relatively thin annular film 92.

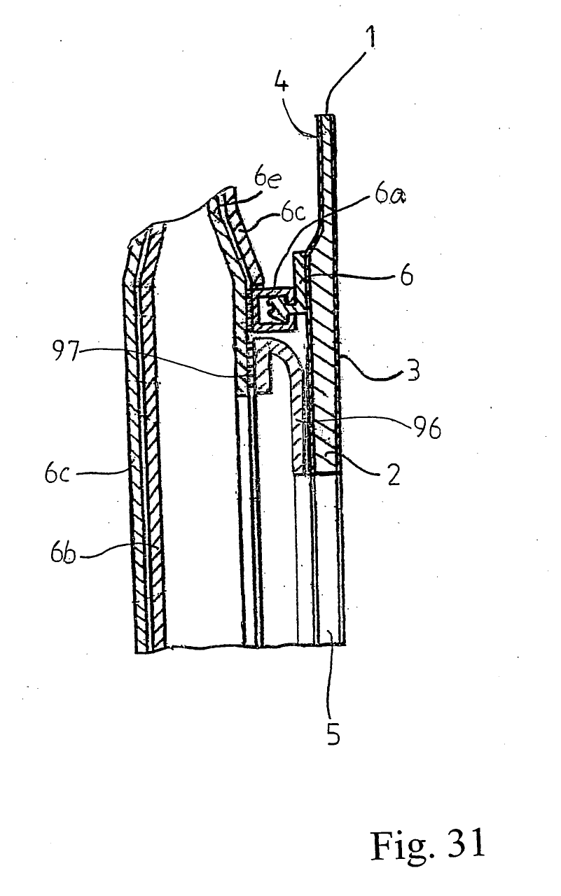

Referring now to FIG. 31, this embodiment is identical to the FIG. 29 embodiment except that the ring 90 with annular film 92 has been substituted by a convoluted ring 96 of a flexible plastic material attached to the proximal bag wall 6e by means of an adhesive or a heat sealing seam 97. The thickness of the ring may vary such that the region near the aperture 5 is thinnest thereby easing the adaptation of the inner edge of the ring 96 to the individual stoma as explained above.

The ostomy bags in the FIGS. 29-31 embodiments are single bag appliances with the bag attached to the face plate by means of interlocking coupling rings. However, the rings 90 and 90′ as well as 96 may just as well be applied to bag in bag appliances such as shown in FIG. 21 and appliances wherein the bag or bags are releasably adhered to the face plate by means of adhesive such as in FIG. 24.

Many variations and modifications are conceivable within the scope of the appended claims.

Thus, the pre-filter ring 65 and 80 may for instance be substituted by a perforated or gas permeable ring of relatively rigid plastic, flexible plastic film or cardboard or other suitable, preferably biodegradable material and preferably provided at the proximal edge region thereof with a flexible annular apron resiliently contacting the distal surface of the face plate between the coupling ring 6 and the stoma aperture 5.

The ring 65 may be configured such that the inner surface facing the stoma is inclined such that the proximal surface of the ring contacting the face plate is radially wider than the distal surface, preferably such that the inner rim of said proximal surface is adjacent the rim of the stoma receiving aperture of the face plate such that the ring 65 itself may perform the “wiping” or “sweeping function described above when the bag or bags are removed.

Instead of attaching the pre-filter ring 65, 80, 83 to the proximal wall 6e of the bag, the ring may be attached to the pouch coupling ring 6a.

The pre-filter rings need not necessarily be attached to the bag wall or the coupling ring, but may be a separate ring that is placed inside the coupling ring 6 prior to attaching the coupling ring 6a to the coupling ring 6.

The location of the venting apertures 20 may be chosen in many manners, but it is preferred that the venting apertures be provided adjacent the top half of the coupling rings such that if the user of the face plate takes a shower without having a bag mounted on the face plate, water from the shower will have more difficulty in penetrating through the venting apertures into the filter pouch and block the filter which preferably also should be placed adjacent the, top of the coupling rings.

The annular film 92 of the FIG. 29 embodiment may be substituted by the sheath 68 of the FIG. 23 embodiment.

It will be obvious to persons skilled in the art that features from one embodiment may be combined with features of another embodiment as long as resulting combination embodiments are within the scope of the appended claims.

For instance, the layer of non-woven material described in connection with FIG. 4A may applied to all the embodiments in FIGS. 1-2 and 4-28.

I claim:

1. An adhesive face plate for securing an ostomy bag to the skin of an ostomy patient, said faceplate comprising:
a flexible plate with a bodyside or proximal surface and a distal surface and comprising a layer of skin friendly adhesive material and a stoma receiving aperture for receiving a stoma of said patient,
generally annular face plate coupling means having an inner and an outer periphery and adapted for fixedly or releasably engaging corresponding bag coupling means of said ostomy bag, said face plate coupling means being secured to said flexible plate with the plane of said face plate coupling means generally parallel to the plane of said flexible plate and generally concentric with said aperture for receiving said stoma,
a filter for de-odorising flatus gas,
one or more flatus gas passages extending between said face plate coupling means and said flexible plate form said inner periphery to said outer periphery and through said filter.

2. A face plate according to claim 1, wherein said face plate coupling means and said bag coupling means are mutually matching coupling rings adapted for releasably engaging one another.

3. A face plate according to claim 1, wherein said face plate coupling means and said bag coupling means are mutually matching surfaces adapted for being releasably or fixedly adhered to one another by means of an adhesive or fixedly adhered to one another by means a heat seal seam.

4. A face plate according to claim 3, wherein one or both of said mutually matching surfaces are provided by a ring of a relatively rigid material.

5. A face plate according claim 1, further comprising a compartment having an exterior portion located outside said outer periphery and an interior portion located between said face plate coupling means and said flexible plate, a flatus gas outlet being provided in a wall of said exterior portion, said compartment forming part of said flatus gas passage, said filter being located within said compartment such that said flatus gas outlet is obstructed by said filter.

6. A face plate according to claim 5, wherein said exterior portion of said compartment is defined by a first film strip of plastic material, preferably adapted to define a pouch.

7. A face plate according to claim 6, wherein said first film strip has a first edge attached to said face plate coupling means and a second edge opposed said first edge attached to said flexible plate so as to allow said flatus gas passage to extend between said first and second edges.

8. A face plate according to claim 7, wherein said first film strip is composed of a second and a third film strip of said plastic material, said second and third film strips comprising said first edge and said second edge, respectively, and said second and third film strips being attached to one another along the edges thereof opposed to said first and second edges, respectively.

9. A face plate according to claim 6, wherein said first film strip has a first edge attached to said coupling ring and a second edge opposed said first edge attached to said flexible plate outside said outer periphery.

10. A face plate according to claim 5, wherein said exterior portion of said compartment extends along part of the circumference of said outer periphery.

11. A face plate according to claim 5, wherein said exterior portion of said compartment extends along the entire circumference of said outer periphery.

12. A face plate according to claim 3, wherein a first edge of a fourth film strip of plastic material is attached to said face plate coupling means and a second edge of said fourth film strip opposed to said first edge is attached to said flexible plate inside said outer periphery, at least one flatus gas inlet aperture provided in said fourth film strip constituting a flatus gas inlet to a corresponding one of said one or more flatus gas passages.

13. A face plate according to claim 12, wherein said fourth film strip is integral with said first film strip.

14. A face plate according to claim 12, wherein said flatus gas inlet aperture is located remote from said gas outlet aperture.

15. A face plate according to claim 14, wherein open cell foam material is arranged in said compartment between said flatus gas inlet aperture and said flatus gas outlet such that substantially the entire cross sectional area of the compartment is filled with said foam material in at least one point between said flatus gas inlet aperture and said flatus gas outlet.

16. A face plate according to claim 12, wherein obstructing means for hindering or preventing stomal fluids and liquids entering said flatus gas passage is provided adjacent said inner periphery.

17. A face plate according to claim 16, wherein said obstructing means comprises a gas permeable, liquid impermeable film arranged such that said film covers the inlet to said flatus gas passage.

18. A face plate according to claim 17, wherein said film is attached to said fourth film strip at a region surrounding said flatus gas inlet aperture.

19. A face plate according to claim 17, wherein said fourth film strip is constituted by said gas permeable, liquid impermeable film.

20. A face plate according to claim 16, wherein said obstructing means comprises a fifth film strip with one longitudinal edge attached to the flexible plate inside said inner periphery along the entire circumference of said coupling ring, the opposed longitudinal edge of said fifth film strip being free to move such that an annular collar extending away from said flexible plate is formed by said fifth film strip.

21. A face plate according to claim 20, wherein the obstructing means comprises a sixth film strip with one longitudinal edge attached to said coupling ring along part of the total circumference thereof and extending inwards from said inner periphery, the opposed longitudinal edge of said sixth film strip being free to move such that a curtain extending generally parallel to the flexible plate is formed by said sixth film strip.

22. A face plate according to claim 21, wherein said inlet to said flatus gas passage is located at said part of the total circumference, preferably near the middle thereof.

23. A face plate according to claim 16, wherein said obstructing means comprises an elongate body of a gas permeable, preferably hydrophobic material such as open cell foam or non-woven material, said body having an arcuate, preferably closed loop and preferably annular shape and being located between said face plate coupling means and said stoma receiving aperture.

24. A face plate according to claim 23, wherein said body abuts said distal surface of said flexible plate along substantially the entire length of said body.

25. A face plate according to claim 23, wherein an annular film is attached to the proximal surface of said body and covers at least part, preferably substantially all the region of said distal surface located between said body and said stoma receiving aperture.

26. A face plate according to claim 25, wherein said annular film extends away from said body beyond the periphery of said stoma receiving aperture such that a stoma received in said stoma receiving aperture will be partly covered by said annular film in the form of a sheath or cuff for said stoma.

27. A face plate according to claim 20, wherein a labyrinth is provided in said compartment between the inlet to said flatus gas passage and said filter such that said flatus gas passage comprises said labyrinth.

28. A face plate according to claim 27 wherein said labyrinth comprises a seventh film strip of plastic material having a first edge attached to said flexible plate together with said second edge of said fourth film strip, said seventh film strip having a second edge strip opposed to said first edge thereof attached to said firm film strip such that the compartment defined between said first film strip and said fourth film strip is subdivided into two chambers, at least one flatus gas flow aperture being provided in said seventh film strip.

29. A face plate according to claim 12, wherein spacing means are provided between said fourth film strip and said first film strip at least adjacent said flatus gas inlet aperture such that a space is provided between said first and fourth film strips to allow flatus gas to flow through said flatus gas inlet aperture unimpeded by any adhesion between said first and fourth film strips.

30. A face plate according to any of the claim 28, wherein spacing means are provided between said fourth film strip and said seventh film strip adjacent said flatus gas inlet aperture such that a space is provided between said first and seventh film strips to allow flatus gas to flow through said flatus gas inlet aperture unimpeded by any adhesion between said seventh and fourth film strips.

31. A face plate according to claim 29, wherein said spacing means comprises a strip of non-woven material.

32. A face plate according to claim 1, wherein said filter is annular and is arranged between said face plate coupling means and said flexible plate.

33. A face plate according to claim 32, wherein said coupling ring is attached to said flexible plate by means of said annular filter.

34. An adhesive face plate for securing an ostomy bag to the skin of an ostomy patient, said faceplate comprising:
a flexible plate with a bodyside or proximal surface and a distal surface and comprising a layer of a skin friendly adhesive material and a stoma receiving aperture for receiving a stoma of said patient,
generally annular face plate coupling means having an inner and an outer periphery and adapted for fixedly or releasably engaging corresponding bag coupling means of said ostomy bag, said face plate coupling means being secured to said flexible plate with the plane of said face plate coupling means generally parallel to the plane of said flexible plate and generally concentric with said aperture for receiving said stoma,
a filter for de-odorising flatus gas,
a flatus gas venting compartment in the form of a pouch or bag made of a first film of plastic material and extending outward beyond said outer periphery, said first film being provided with a flatus gas outlet aperture, and
a flatus gas passage extending from said inner periphery into the interior of said venting pouch and through both said filter and said flatus gas outlet aperture, said filter being arranged such that substantially all said flatus gas vented through said flatus gas aperture flows through said filter.

35. A face plate according to claim 34, wherein said face plate coupling means and said bag coupling means are mutually matching coupling rings adapted for releasably engaging one another.

36. A face plate according to claim 34, wherein said face plate coupling means and said bag coupling means are mutually matching surfaces adapted for being releasably or fixedly adhered to one another by means of an adhesive or fixedly adhered to one another by means of a heat seal seam.

37. A face plate according to claim 34, wherein said flatus gas passage extends between said face plate coupling means and said flexible plate.

38. A face plate according to claim 34, wherein said flatus gas passage extends at least partly through a conduit extending through a portion of said face plate coupling means from a conduit inlet to a conduit outlet.

39. A face plate according to claim 38, wherein one edge of said venting pouch is attached to a body side or proximal surface of said face plate coupling means facing said flexible plate and the opposed edge of said venting pouch is attached to a distal surface of said face plate coupling means facing away from said flexible plate, said conduit outlet being located between said proximal and distal surfaces such that flatus gas exiting from said conduit enters said venting pouch.

40. A face plate according to claim 34, wherein said pouch extends along part of the circumference of said outer periphery.

41. A face plate according to claim 34, wherein said pouch extends along the entire circumference of said outer periphery.

42. A face plate according to claim 34, wherein a first edge of a first film strip of plastic material is attached to said coupling ring and a second edge of said first film strip opposed to said first edge is attached to said flexible plate inside said outer periphery, at least one flatus gas inlet aperture being provided in said first film strip.

43. A face plate according to claim 34, wherein open cell foam material is arranged in said pouch upstream of said filter such that substantially the entire cross section area of said venting pouch is filled with said foam material at at least one point.

44. A face plate according to claim 34, wherein obstructing means for hindering or preventing stomal fluids and liquids entering said flatus gas passage is provided adjacent said inner periphery.

45. A face plate according to claim 44, wherein said obstructing means comprises a gas permeable, liquid impermeable film arranged such that said film covers the inlet to said flatus gas passage.

46. A face plate according to claim 44, wherein said film is attached to said first film strip at a region surrounding said flatus gas inlet aperture.

47. A face plate according to claim 45, wherein said first film strip is constituted by said gas permeable, liquid impermeable film.

48. A face plate according to claim 44, wherein said obstructing means comprises a second film strip with one longitudinal edge attached to the flexible plate inside said inner periphery along the entire circumference of said coupling ring, the opposed longitudinal edge of said second film strip being free to move that an annular collar extending away from said flexible plate is formed by said second film strip.

49. A face plate according to claim 44, wherein the obstructing means comprises a third film strip with one longitudinal edge attached to said coupling ring along part of the total circumference thereof and extending inwards from said inner periphery, the opposed longitudinal edge of said third film strip being free to move such that a curtain extending generally parallel to the flexible plate is formed by said third film strip.

50. A face plate according to claim 49, wherein said inlet to said flatus gas passage is located at said part of the circumference, preferably near the middle thereof.

51. A face plate according to claim 44, wherein said obstructing means comprises an elongate body of a gas permeable.

52. A face plate according to claim 51, wherein said body abuts said distal surface of said flexible plate along substantially the entire length of said body.

53. A face plate according to claim 51, wherein an annular film is attached to the proximal surface of said elongate body and covers at least part of the region of said distal surface located between said elongate body and said stoma receiving aperture.

54. A face plate according to claim 53, wherein said annular film extends away from said body beyond the periphery of said stoma receiving aperture such that a stoma received in said stoma receiving aperture will be partly covered by said annular film in the form of a sheath or cuff for said stoma.

55. A face plate according to claim 34, wherein a labyrinth is provided in said pouch between the inlet to said flatus gas passage and said filter such that said flatus gas passage comprises said labyrinth.

56. A face plate according to claim 55, wherein said labyrinth comprises a fourth film strip of plastic material arranged and adapted to subdivide said pouch into two chambers, one chamber communicating directly with said flatus gas passage inlet and the other chamber containing said filter, at least one flatus gas flow aperture being provided in said fourth film strip.

57. A face plate according to claim 38, wherein spacing means are provided at least adjacent said conduit outlet such that a space is provided between the film walls of said pouch to allow flatus gas to flow through said flatus gas conduit unimpeded by any adhesion between said film walls.

58. A face plate according to claim 42, wherein spacing means are provided at least adjacent said flatus gas inlet aperture such that a space is provided downstream from said flatus gas inlet aperture to allow flatus gas to flow through said flatus gas inlet aperture unimpeded by any adhesion between said first film strip and a pouch wall or the flexible plate.

59. A face plate according to claim 57, wherein said spacing means comprises a strip of non-woven material.

60. A face plate according to claim 34, wherein said proximal surface of said flexible plate is convex, a relatively rigid convex ring being attached to said distal surface of said flexible plate.

61. A face plate according to claim 60, wherein flatus gas passages are provided through said convex ring.

62. An ostomy appliance for receiving human stomal discharge comprising in combination an adhesive face plate according to claim 34 and an ostomy bag for receiving human stomal discharge releasably or fixedly attached to said face plate.

63. An ostomy appliance according to claim 62, wherein said ostomy bag comprises a single bag of a gas and liquid impermeable material.

64. An ostomy appliance according to claim 62, wherein said ostomy bag comprises two bags, an outer bag of a gas and liquid impermeable material and an inner bag of a flexible material and contained within said outer bag.

65. An ostomy appliance according to claim 63, wherein said elongate body is attached to said ostomy bag and or said bag coupling means such that removal of said ostomy bag from said face plate entails removal of said elongate body from said face plate.

66. An ostomy appliance according to claim 64, wherein said elongate body is attached to said inner ostomy bag such that removal of said inner ostomy bag from said face plate entails removal of said elongate body from said face plate.

67. A method of cleaning an adhesive face plate for securing an ostomy bag having an opening for receiving stomal discharge to the skin of an ostomy patient, said faceplate comprising:
a flexible plate with a bodyside or proximal surface and a distal surface and comprising a layer of a skin friendly adhesive material and a stoma receiving aperture for receiving a stoma of said patient, and
generally annular face plate coupling means having an inner periphery generally concentric with and spaced from the edge of said aperture for receiving said stoma, a generally annular face plate distal surface being defined between said inner periphery and said edge, said face plate coupling means being adapted for releasably engaging corresponding bag coupling means of said ostomy bag such that said bag opening is in register with said face plate aperture,
the method comprising the steps of:
providing a separate closed loop, preferably annular elongate body having dimensions allowing said body to be located within said inner periphery and abutting said annular face plate surface,
attaching said body to said ostomy bag,
attaching said ostomy bag to said face plate by engaging said face plate coupling means with said bag coupling means such that said body is located within said inner periphery and abutting said annular face plate surface, and
removing said ostomy bag together with said body by disengaging said face plate coupling from said bag coupling means.

68. A method according to claim 67, wherein said face plate coupling means and said bag coupling means are mutually matching coupling rings adapted for releasably engaging one another.

69. A method according to claim 67, wherein said face plate coupling means and said bag coupling means are mutually matching surfaces adapted for being releasably or fixedly adhered to one another by means of an adhesive or fixedly adhered to one another by means a heat seal seam.

70. A method according to claim 67, wherein said body is attached to said bag by means of adhesive or heat sealing at an annular region adjacent to said bag opening.

71. A method according to claim 67, wherein said body is attached to said bag by means of adhesive or heat sealing to said bag coupling means.

72. A method according to claim 67, wherein said body comprises a ring of shape recoverable, compressible, preferably hydrophobic material such as open cell or closed cell foam material or of non-woven material or any other material having similar properties.

73. A method according to claim 67, wherein said body abuts substantially the entire area of said annular face plate surface along substantially the entire length of said body.

74. A method according to claim 72, wherein an annular film is attached to the proximal surface of said body and covers at least part of the area of said annular face plate surface located between said body and said edge of said face plate aperture.

75. A method according to claim 74, wherein said annular film extends away from said body beyond said edge such that a stoma received in said stoma receiving aperture will be partly covered by said annular film in the form of a sheath or cuff for said stoma.

76. An ostomy appliance for receiving human stomal discharge from an ostomy patient and comprising in combination an adhesive face plate with a stoma receiving aperture for receiving a stoma of said patient and an ostomy bag with an opening for receiving said stomal discharge for receiving said stomal discharge releasably attached to said face plate,
said face plate comprising a flexible plate having a bodyside or proximal surface and a distal surface, a layer of a skin friendly adhesive material, and generally annular face plate coupling means having an inner periphery generally concentric with and spaced from the edge of said aperture for receiving said stoma, a generally annular face plate distal surface being defined between said inner periphery and said edge, said face plate coupling means being adapted for releasably engaging corresponding bag coupling means of said ostomy bag such that said bag opening is in register with said face plate aperture, the ostomy appliance further comprising:
a separate closed loop, elongate body attached to said ostomy bag and located within said inner periphery and abutting said annular face plate surface when said face plate coupling means are engaged with said bag coupling means.

77. An ostomy appliance according to claim 76, wherein said face plate coupling means and said bag coupling means are mutually matching coupling rings adapted for releasably engaging one another.

78. An ostomy appliance according to claim 76, wherein said face plate coupling means and said bag coupling means are mutually matching surfaces adapted for being releasably or fixedly adhered to one another by means of an adhesive or fixedly adhered to one another by means a heat seal seam.

79. An ostomy appliance according to claim 76, wherein said body is attached to said bag by means of adhesive or heat sealing at an annular region adjacent to said bag opening.

80. An ostomy appliance according to claim 76, wherein said body is attached to said bag by means of adhesive or heat sealing to said bag coupling means.

81. An ostomy appliance according to claim 76, wherein said body comprises a ring of shape recoverable, compressible.

82. An ostomy appliance according to claim 76, wherein said body abuts substantially the entire area of said annular face plate surface along substantially the entire length of said body.

83. An ostomy appliance according to claim 81, wherein an annular film is attached to the proximal surface of said body and covers at least part, preferably substantially all the area of said annular face plate surface located between said body and said edge of said face plate aperture.

84. An ostomy appliance according to claim 83, wherein said annular film extends away from said body beyond said edge such that a stoma received in said stoma receiving aperture will be partly covered by said annular film in the form of a sheath or cuff for said stoma.

85. An ostomy appliance according to claim 81, wherein the ring is of a hydrophobic material.

86. An ostomy appliance according to claim 85, wherein the hydrophobic material is one of a group of open cell foam material, closed cell foam material, and a non-woven material.

87. A face plate according to claim 51, wherein the gas permeable material is a hydrophobic material.

88. A face plate according to claim 87, wherein the hydrophobic material is one of a group of an open cell foam material, a closed cell foam material, and a non-woven material.

89. A face plate according to claim 51, wherein the elongate body has an annular shape and is located between the face plate coupling means and the stoma receiving aperture.

90. A face plate according to claim 53, wherein the annular film covers substantially all of the region of the distal surface located between the elongate body and the stoma receiving aperture.
